SEQUENCE LISTING (6 sequences)

SEQ ID NO: 1 - protein, 1294 aa, Plasmodium Falciparum
MKVGIIFFCLFFFVVLGACNNVKERIFKNIKKRTKFIILNEPIVDLSFSENLFHTLLFDLDVDKNLYTLDESLLNLENLNYSSIFRLLVDTYKNIKENEDDNKNIRYIFLGTSFSRIHPLNFEYFLRKLNKYIYNGNIYEKGNVDIRGILEEYNKEIEEKKLEKQKLNKIKDKNNNNNNNNNSKFSKDGDNEDFNNKNDLYNPSDKLYNNNDDIDVHELLEEIITKEKRFFLNDDDDNDSNDKYILKTDEVNKYKGFFIGYGFNDDIPSVIHHYNFDKNFLFPSLNSGIILDITLLKNIYEVSNILLSNNEKDQSIHIDYIYEVTKYIKENLRVRLTHSENVCLNEEQNIHLLDNDPNNFEIYKYYQVLNLFKDYNKNTEEKQYEKIGHENVRHEETSSEGNENLNRNTKHNNDNNNDNNNYSEDAIAELLLSYFNVFYPISTCMCYSIRSKHESLMDYDKYHMINLENDIKLKHYIKETEEIHFNSIEEYKMKLNRINYKYDTLLEEHENLVTHKNILIGIKTSINTEEERIPHIKNTYDNKENTQIIFNTFNYDNKLKEKNTFGFYNNSLLQNALENDNIDLDIIYMSDKESQKYDNLYFNSKVTSKEGLCEKLKHMIYYYYEEYVMKNSEKKYFFIADDDTFVNVKNLIDVTNLTLNTCSHSKKYMYDKYIKSYDFVKENEALFLQNFPKKTLFLYSYLKDTFAKTIQTLKKYDYVPKYCQGGILSKKHKNNDSDDDHDHHVGNKQNNDSTNHQDIEKNQVNVINNNNNNNNNKAKSIPIYLGRRYSYNTFSTNSNEYFYDYLTGGAGILINDETAKRIYECKECTCPSTNSSMDDMIFGKWAKELGILAINFEGYFQNSPLDYNKKYINTLVPITYHRLNKNRTTKESRDMYFNYLVNYNRNDKEQNKDIYVDYLDRNHKNMIDNVFHYFFYVNMYDEKNKVVTKIEHNADMNSKKNKSKNPQKLNNTQGDKNVNDDENVNDDENVKGDENVKGDENVKGDEYMKGDENVKGDENVKDDENVKDDENIKGDDNNYNVDNMENIDDIINMVESVDDDVMERNKKGTGKEKKDDKNHNNKEKATDVKKSSVPTNNIDKNEDTTKYVIKMNEKIYNRMQESGKYKQLFDINKFFKKEIEGHPYFQKIKKKNEKAKKEKEKMNQLKKQKDYTNNYFHTSNMQGNFNQQKMGNYQNQENEENDFFDQRPEIEEDAINPMDYEEYMENLSNFEDDGEPYDEYDDYDDFVNTINADKLKINDQNKHLYEQIKDIAQPPVNFQNDQNSNTFDFDTDEL

SEQ ID NO: 2 - protein, 408 aa, Plasmodium Falciparum
MLLFFAKLVVFTFFFWLLKYGKTRSYPKSGHKGHTKLNQPVVRTLADFNDMFANQKNTFNFLKHINHYKNEQDTNNTHTPNHDEYSHNLPKNHEESNANMNNHNSFNDKSVNKKEAFDQFLQTLLNNYEIMHKEDESKESNQHNYKEGPSYEDKKNMYKEILKGYYNVFFENYANDTESNVHNKPEEVHKHEEIHKHRKLHKHEEVHKPEEFHKPEEFHKHEKVHKHEEVHKPEEVHKHEENHKHEENHKPQMVGQAPPEKEIRQESRTLILGSFPQAGEILREDLWNKEDNKFSYALDPNDYASIEDKLLGSIFGYFKKNHDNLVKHLLQQINTYKHKYMELKEQYINEVMKLKKIYNKSIMVIFIASCISILGPVMLHMHQNNPEEFFATILSFSISLGLHNLLLT

SEQ ID NO: 3 - DNA, 3885 bp, Plasmodium Falciparum
atgaaggttggaattatatttttttgtttatttttttttgtggttcttggagcgtgtaacaatgtgaaggaaaggatttttaagaatattaaaaaaagaaccaaatttattatattgaatgagcccatagtagatttaagttttagtgagaatttatttcatactttattatttgatttagatgtagataagaatttatatacattggatgagagtttattaaatcttgagaacttgaattattcctcaatatttcgtttacttgttgatacctataagaatataaaagaaaatgaagatgataataaaaatattcgatatatatttttaggtacatcgttttcacgtattcatcccttaaattttgaatattttttgagaaagctgaacaaatatatatataatgggaacatatatgaaaagggtaatgtggatatcagaggaatattggaagaatataataaggagattgaagagaagaagctagaaaaacaaaaactgaacaagatcaaagataagaataataataataataataataataatagtaaattttctaaagatggtgataatgaagactttaataataagaatgatttgtacaatccatcggataaattatacaataataatgatgatatcgatgtacatgaactattagaagagattattacaaaagaaaaaaggtttttcttaaacgatgatgatgataatgatagtaatgataaatatatattaaaaactgacgaggttaataaatataaaggattttttataggatatggttttaatgatgatataccatcagtaattcatcattataattttgataagaactttttatttccttctttaaatagtggtattatattagatataacattattaaaaaatatatatgaagtttctaatatattattatcgaataatgaaaaggatcaatctattcatatagattatatttatgaagttacaaaatatataaaagaaaatttaagagtacgtttaacacattccgaaaatgtatgtttaaacgaagaacaaaatattcatttattagataatgatcctaataatttcgaaatatataaatattatcaagtgctgaacttatttaaagattataataagaatacagaagaaaagcaatatgaaaaaattggccatgaaaatgttagacatgaagaaacatcatctgaaggtaatgaaaaccttaatagaaataccaaacataataatgataataataatgataataataattatagtgaagatgcgattgccgaattacttctctcctattttaatgtgttctatccaatatctacatgtatgtgctattcaataagatcaaaacatgaatccctaatggattatgataaatatcatatgatcaatttagaaaacgatataaaattaaaacattatataaaagaaacagaagaaatacattttaatagtattgaagaatataaaatgaaacttaatcgtattaattataaatatgatactttattagaagaacatgaaaatttagtaacacataaaaatatactcataggtataaaaacaagtataaatacagaagaagaaagaattccacatattaaaaatacatatgataataaagaaaatacacaaataatattcaatacattcaactatgataataaattaaaagaaaaaaatacatttggattttataataattcccttttacaaaatgctttagaaaatgataatatagatttagatattatctatatgtctgataaggaaagccaaaaatatgataatttatattttaattctaaagtaacatcaaaagaaggcttatgtgaaaaattaaaacatatgatatattattattatgaagaatatgttatgaaaaattcagaaaaaaaatatttctttattgcagatgatgatacttttgttaatgtaaaaaatttaatagatgtaacaaatttaacattaaatacttgttcacattctaaaaaatatatgtatgataaatatatcaaatcttatgattttgttaaagaaaatgaagccttatttcttcaaaattttccaaaaaaaactttatttctttattcctatttgaaagatacctttgccaaaactatacaaaccttgaagaaatatgactatgttcctaaatattgtcagggtggtatcctatcaaaaaaacataaaaataatgatagtgatgatgatcatgatcatcacgtgggtaataaacaaaataatgatagtacgaatcatcaagatattgaaaaaaatcaagtaaatgtaataaataataataataataataataataataaagcaaaatccatacctatatacttaggaagaagatattcatataatacattttctacaaattcaaatgaatatttttatgattatttaactggaggtgctggtattttaattaatgatgaaacagctaaacgaatatatgaatgcaaagaatgcacatgcccatcaacaaattcctcaatggatgatatgatatttgggaaatgggctaaagaattaggaattttagccataaactttgaaggatattttcaaaactccccacttgattataacaaaaaatatattaatactcttgtacctattacatatcatagattaaataaaaatagaacaaccaaagaatcaagagatatgtattttaattatctagtaaattataatagaaatgataaagaacaaaataaagacatatatgttgattatctagatagaaatcataaaaatatgatagataatgtattccattactttttttatgtaaatatgtatgatgaaaaaaataaagtcgtcaccaaaattgagcacaatgctgatatgaacagtaaaaagaataaatcaaagaacccacaaaaattaaataatactcaaggggacaaaaatgtaaatgatgatgaaaatgtaaatgatgatgaaaatgtgaaaggtgatgaaaatgtgaaaggtgatgaaaatgtgaaaggtgatgaatatatgaaaggtgatgaaaatgtgaaaggtgatgaaaatgtgaaagatgatgaaaatgtgaaagatgatgaaaatataaaaggtgatgataataattacaatgtggataatatggaaaacatagatgatattattaatatggttgaaagcgttgatgatgatgttatggaacgtaacaaaaaaggaacgggtaaagaaaaaaaggatgataagaatcataataataaagaaaaagctaccgatgtgaaaaaatcaagtgtacctactaataatatagataaaaatgaagacactacaaaatatgtcataaaaatgaatgaaaaaatttataatagaatgcaagaaagtggtaaatacaaacaattattcgatataaataaatttttcaaaaaagaaatcgaaggacatccttattttcaaaaaataaaaaaaaagaatgaaaaggccaaaaaagaaaaagaaaaaatgaatcaattaaaaaaacaaaaggattatacaaataattatttccatacatcaaatatgcagggaaattttaatcaacaaaaaatgggaaactatcaaaatcaagagaatgaagaaaatgatttttttgatcaacgtcctgaaatagaagaagatgcaattaatccaatggattatgaagaatatatggaaaatttatcaaattttgaagatgatggcgaaccatatgacgaatatgatgattatgatgatttcgtaaatacaattaatgcagataaattaaaaattaatgatcaaaataaacacttatatgaacaaatcaaagatatagcgcaaccacctgttaatttccaaaatgatcaaaattcaaatacttttgattttgacacagatgagttgtaa

SEQ ID NO: 4 - DNA, 1120 bp, Plasmodium Falciparum
atgttactcttttttgcaaaacttgtcgtatttacctttttcttttggcttttaaaatatgggaaaacgaggtcatatcccaaatctggccataagggacatacgaaattaaatcaaccagtagttagaacattagcagattttaatgacatgtttgcaaaccaaaaaaatacatttaattttctaaaacatataaatcattataaaaatgaacaagatacaaataatacacacacgccaaatcatgatgaatattctcataatttgccaaaaaatcacgaagagtcaaatgcaaatatgaacaatcataattctttcaatgacaaatctgttaataaaaaagaagctttcgatcaatttttacaaacgttattaaacaattatgaaataatgcataaagaagatgaaagtaaagaatcaaatcaacataactataaagaaggtccctcatatgaagataaaaaaaatatgtacaaagaaatattgaaaggatattataatgtattttttgaaaattatgcaaacgacacagaatcaaatgtacataataaacctgaggaagttcataaacatgaggaaattcataaacataggaaacttcataaacatgaagaagttcataaacctgaggaatttcataaacctgaggaatttcataaacatgagaaagttcataaacatgaagaagttcataaacctgaggaagttcataaacatgaggaaaatcataaacatgaggaaaatcataaacctcaaatggtaggtcaagcacctccagaaaaagagatacgccaagaatcaagaactctaatacttggttcatttccccaagcaggtgaaatattaagagaggatttatggaacaaagaggataacaaatttagttacgcacttgaccctaatgattatgcatctatagaagataaacttttaggatctatatttggatactttaaaaaaaatcatgacaatttggttaaacatttgttacaacaaattaatacttacaaacataaatatatggaacttaaagaacaatatattaatgaagttatgaaacttaaaaaaatatataacaaaagcatcatggtcatatttatagcatcttgtatttcaatat

SEQ ID NO: 5 - protein, 30 aa, Plasmodium Falciparum
NNSKFSKDGDNEDFNNKNDLYNPSDKLYNN

SEQ ID NO: 6 - protein, 33 aa, Plasmodium Falciparum
YEIMHKEDESKESNQHNYKEGPSYEDKKNMYKE

(19) United States
(12) Patent Application Publication
(10) Pub. No.: US 2004/0202667 A1
(43) Pub. Date: Oct. 14, 2004

(54) Antigens and their use as diagnostics and vaccines against species of plasmodium

(76) Inventors: Daniel Carucci, Washington, DC (US); John Yates, La Jolla, CA (US); Laurence Florens, La Jolla, CA (US); Yimin Wu, Rockville, MD (US)

Correspondence Address: NAVAL MEDICAL RESEARCH CENTER, ATTN: (CODE 00L), 503 ROBERT GRANT AVENUE, SILVER SPRING, MD, 20910-7500, US

(21) Appl. No.: 10/377,636

(22) Filed: Mar. 4, 2003

Related U.S. Application Data
(60) Provisional application No. 60/361,282, filed on Mar. 4, 2002.

(51) Int. Cl. C12Q 1/68; A61K 39/00; A61K 39/38

(52) U.S. Cl. 424/184.1

(57) ABSTRACT

Two proteins and their use as substrates for vaccines intended to initiate an immune response in a mammalian subject against infection with species of Plasmodium for use in the diagnosis of Plasmodium infection and for their use in the development of antimalarial drugs. This invention also relates to the diagnostic, isolation and purification assays based on these Plasmodium proteins. This invention further relates to immunological reagents, specifically antibodies directed against these Plasmodium proteins.

### CROSS REFERENCE TO RELATED APPLICATIONS

This application claims priority to U.S. Provisional Application No. 60/361,282 filed Mar. 4, 2002.

### FIELD OF THE INVENTION

This invention relates specifically to two genes encoding Plasmodium falciparum proteins, methods for the detection of these and similar proteins located on the surface of Plasmodium infected mammalian cells, and vaccines for the protection against malaria in humans and non-human mammals. This invention further relates to the diagnostic, isolation and purification assays based on these Plasmodium proteins. This invention further relates to immunological reagents, specifically antibodies directed against these Plasmodium proteins.

### DESCRIPTION OF THE PRIOR ART

The disease Malaria is caused by infection with one of four species of Plasmodium: P. falciparum, P. vivax, P. malariae and P. ovale. Plasmodium parasites belong to the family Apicomplexa and are eukaryotic protozoan parasites that possess a complex life cycle which involves both an invertebrate host (Anopheles mosquito) and a mammalian host. The parasite life cycle includes direct inoculation into the mammalian host by the bite of an infected Anopheles mosquito which injects stages of the parasite known as “sporozoites”. The sporozoites rapidly invade cells of the liver by an active invasion process which is thought to involve attachment to the liver cells and which involves a cascade of processes which results in the parasite taken up residence inside a liver cell (hepatocyte) (Hollingdale, McCormick et al. 1998). The parasite undergoes asexual multiplication over a period of several days resulting in production of thousands of parasites which are released into the host circulation. These “merozoite” forms invade host cell erythrocytes (red blood cells) by an active process which involves attachment to the exterior surface of the erythrocyte, reorientation, and invagination (in folding) of the erythrocyte membrane until the parasite is completely enveloped by the erythrocyte (Preiser, Kaviratne et al. 2000). While inside the erythrocyte the parasite begins to grow using the erythrocyte hemoglobin as an energy source and divides into approximately one dozen additional parasites. During this growth phase, some of the Plasmodium proteins are exported to the surface of the erythrocyte and can be found associated with the erythrocyte membrane. Some of these proteins are thought to represent important targets for vaccine development as their location allows them exposure to the host immune system (Chen, Fernandez et al. 1998). Two models are often used to describe the development of immunity to malaria and as a tool for the development of new strategies for malaria vaccine development (Richie and Saul 2002):

Irradiated Sporozoite Model

Naturally Acquired Immunity (NAI)

(a) The irradiated sporozoite model: This model involves immunizing volunteers via the bites of irradiated Plasmodium-infected Anopheles mosquitoes. The parasites within the mosquitoes are damaged but not killed by the radiation, and thus constitute an attenuated whole organism vaccine. They are able to enter the blood stream of vaccinees while the mosquitoes feed, invade liver cells, and undergo limited development, but cannot progress to the pathogenic blood stages due to the attenuation caused by radiation. While undergoing development in the liver, these damaged parasites induce a strong protective immune response directed against liver stage parasites. As mentioned above, it appears that this strong protective immunity represents the sum of many immune responses directed at a variety of antigens derived from the whole organism attenuated sporozoite vaccine. When batches of irradiated, infected mosquitoes are allowed to feed on volunteers over a 6-month period, the level of immunity develops sufficiently to protect at least 95 percent of the human volunteers tested when subsequently challenged with intact parasites. The immunity lasts for at least 9 months and is not strain-specific (but does appear to be species-specific). If that level of immunity could be reproduced with a subunit vaccine, it would be considered very effective because all manifestations of disease would be prevented. Because this immunity is based on liver stage (pre-erythrocytic) immunity, it forms a model for pre-erythrocytic stage vaccines designed to completely prevent malaria infection.

(b) The naturally acquired immunity (NAI) model: This model is based on studies of children and adults living in malaria-endemic areas. It has been noted that if children who live in malaria endemic areas survive and reach the age of 10, they remain susceptible to infection with malaria parasites, but do not develop severe disease or die of malaria. In other words, they are protected through acquired immunity against severe disease and death due to malaria infection. This immunity persists for the rest of their lives as long as they continue to live in the malarious area. They may continue to be re-infected with parasites, as shown in cleared-cohort studies, but their health will not be significantly affected by the parasites. NAI limits the number of parasites in the blood and reduces their clinical effect on the host. Because this immunity is based on blood stage antigens, it forms a model for erythrocytic stage vaccines designed to curtail disease and death, even if not preventing infection.

It has been well established that protective immunity against malaria infection is mediated, in part, by circulating antibodies (Mohan and Stevenson 1998). Passive transfer of hyperimmune antibodies obtained from one geographical location can protect against malaria infection in other regions, indicating that the target antigens may be highly conserved among diverse parasite strains (McGregor 1963; McGregor and Wilson 1988). These conserved antigens, located on the surface of parasite-infected erythrocytes thus accessible to protective antibodies, are good vaccine candidates and yet to be identified. A conventional approach to identify surface antigens is to use hyperimmune sera from individuals living in endemic regions (Howard 1988; Fernaders 1998; Kyes 1999). Two surface antigens identified so far by this method, PfEMP1 proteins and Rifins, are highly variable and their roles in the humoral immune protection are still under investigation. In addition, the approach is limited to the identification of highly immunogenic or abundant molecules.

The completion of P. falciparum genome sequencing project, combined with advanced proteomics technologies and bioinformatics tools, has allowed the profiling of expressed parasite proteins to be carried out in an unprecedented scale, with higher sensitivity and efficiency (Florens, Washburn et al. 2002). The advantage of MudPIT technology, a two-dimensional liquid chromatography coupled with tandem mass spectrometry, is its ability to analyze complex protein mixture, particularly, membrane protein mixture that is difficult resolve in other gel-based protein separation systems (Eng, McCormack et al. 1994; Washburn, Wolters et al. 2001).

Development of vaccines against malaria is focused on the identification of parasite proteins found to be present at a particular stage of the parasites life cycle, the design and construction of a vaccine delivery system which is meant to stimulate the desired immune response against that identified protein and which is meant to eliminate, disable or interrupt the function of the parasite within the host(s).

A key component of this vaccine strategy is the identification of proteins at particular stages of the parasite life cycle. Recently, an approach has been developed and applied to the identification of Plasmodium proteins from isolated stages of the parasite life cycle. This approach which employs microcapillary liquid chromatography coupled with tandem mass spectrometry has resulted in the identification of over 2,500 Plasmodium proteins from several stages of the parasite life cycle (Florens, Washburn et al. 2002). Some of these proteins represent potential targets of new malaria vaccines.

At present, there are no licensed vaccines against malaria. The most effective malaria vaccine that would result in sterilizing protective immunity would be directed toward eliminating the parasite while inside the liver cells. However, vaccines that are designed to reduce the number of circulating and sequestered parasites from the mammalian host blood stream would result in a substantial reduction in morbidity and mortality, especially in children and pregnant women living in areas of malaria transmission. This type of vaccine would mimic the naturally acquired immunity that develops over years of exposure to blood stage parasites living and circulating in the host blood stream. It would also be a vaccine which is directed toward parasite proteins expressed either by the circulating parasites before invasion into red blood cells, or to those parasite proteins expressed on the surface of the red blood cell. The most well characterized protein expressed on the surface of P. falciparum infected red blood cells, PfEMP1 (or variant surface antigen) has been shown actually to represent a large family of diverse proteins and has been shown to stimulate immune responses that can reduce parasite numbers in the circulation. The diversity of this protein within the parasite genome and its role in “antigenic switching” may limit its role in providing long-term protection against P. falciparum. There is a need to identify additional Plasmodium proteins on the surface of infected erythrocytes for the development of vaccines directed against these proteins. It is of further interest to develop diagnostic tests for the presence of Plasmodium infections in mammals. To date, the most reliable diagnostic test and the one that is the gold standard used in clinical laboratories is the examination of blood for the presence of parasites by Giemsa staining methods. This method, however, requires a skillful microscopist who has been trained in the identification of malaria parasites within red blood cells. In many areas of the world where malaria is highly endemic, there are an abundance of skilled microscopists who are adept at reading Giemsa stained blood films. However, in the US and other industrialized nations where malaria infection in humans is not abundant, misdiagnosis of malaria due to the absence of trained microscopists can result in a delay in providing adequate treatment and potential death in those infected. The development of a highly sensitive and reliable in vitro assay to detect the presence of Plasmodium in the blood would likely reduce the rate of misdiagnosis and likely result in prompt and appropriate treatment. The identification of parasite proteins expressed in the blood stage of Plasmodium would form the foundation for the development of a clinical assay for Plasmodium in humans and other mammals. Finally, development of new antimalarial drugs may be accelerated by the identification of Plasmodium parasite proteins and their association with biochemical and signal transduction pathways. Parasite proteins expressed at the surface of red blood cells may provide a link to parasite residing within to the external environment. These proteins may therefore represent components of a signal transduction pathway to which directed interruption either by drug or small molecule could result in the parasite receiving misinformation to its detriment and potential death.

### SUMMARY OF THE INVENTION

It is an object of this invention to identify two Plasmodium falciparum proteins expressed at the surface of infected erythrocytes in humans.

It is another object of this invention to use these proteins singly or together as vaccines, either as native or recombinant proteins or peptides.

It is another object of this invention to use the genes encoding these proteins as nucleic acid vaccines or in recombinant viruses, or other vaccine delivery systems whose intent is to generate an immune response in the recipient against these proteins.

It is another object of this invention to use either the native or recombinant protein or peptide vaccines in combination with nucleic acid, recombinant viral vaccines or other delivery systems whose intent is to generate an immune response in the recipient against these proteins.

It is another object of this invention to use these proteins or genes encoding these proteins to detect the presence of Plasmodium parasites in the blood or tissues of human or mammals. It is another object of this invention to use these proteins or genes encoding these proteins in the development of drugs or small molecule interventions designed to interrupt metabolic or signaling pathways in Plasmodium.

It is another object of this invention to identify the orthologous proteins or genes encoding these proteins from Plasmodium where the species is P. vivax, P. ovale or P. malariae. These and additional objects of the invention are accomplished by identifying the presence of these proteins associated with the erythrocyte membrane in Plasmodium infected red blood cells or in the case of other species of Plasmodium (P. vivax, P. ovale or P. malariae) orthologous sequences based on sequence similarity comparisons using, for example, the computer program BLAST (Altschul SF et al) to identify proteins of similar primary amino acid sequence or genes of similar nucleic acid sequence. The detection of the proteins associated with the erythrocyte membrane is accomplished by the purification of erythrocyte membrane proteins from infected in vitro culture of P. falciparum using an affinity purification system and subjecting these purified proteins to liquid capillary/tandem mass spectrometry or multidimensional protein identification technology (MudPIT) to generate mass spectral patterns. These mass spectral patterns can be used to search computer databases for predicted mass spectral patterns of known or predicted proteins. When potential proteins are identified and represent Plasmodium proteins expressed in association with erythrocyte membrane, they are subjected to further verification of location by protein chemistry and immunological means. These means would include the production of protein-specific antisera in animals by immunization with native or recombinant protein, peptide, nucleic acid, recombinant virus or other means and the use of these antisera in immunolocalization by confocal microscopy, Immunofluorescence antibody testing, immunoelectron microscopy or other methods to localize the protein within or in association with the host cell. We have used these methods to identify two proteins from Plasmodium falciparum which are associated with the infected human erythrocyte. The proteins, designated PfSA1 for Plasmodium falciparum surface antigen 1 and PfSA2 for Plasmodium falciparum surface antigen 2 have been shown to be associated with the P. falciparum erythrocyte membrane but not from uninfected erythrocytes using antisera raised in mice to peptides derived from each protein by immunolocalization using confocal microscopy. We have further shown that these proteins are associated in part at the exterior surface of infected erythrocytes by demonstrating that exposure of whole infected erythrocytes to trypsin and chymotrypsin which digests proteins at the erythrocyte surface but not within the erythrocyte abolishes the reactivity of the mouse antisera to the infected erythrocytes and is further supported with the demonstration that inclusion of inhibitors to trypsin and chymotrypsin can prevent this abolished reactivity.

It is also a feature and advantage of the inventive subject matter to provide potential new vaccine target antigens that would stimulate an immune response to Plasmodium infected erythrocytes and result in clearance from the body of these parasites, limit the parasite's ability to replicate inside the host and limit the clinical disease caused by the parasite or as the result of the parasite residing in the host and host cells.

It is also a feature and advantage of the inventive subject matter to identify drugs or small molecules that would associate with or interact with these proteins causing an alteration in the parasite biological function and which would be deleterious to the survival of the parasite inside the host or interrupt the parasite life cycle.

The foregoing and other features and advantages will become further apparent from the following detailed description of the presently preferred embodiments, when read in conjunction with the accompanying examples and made with reference to the accompanying drawings. It should be understood that the detailed description and examples are illustrative rather than limitative, the scope of the present invention being defined by the appended claims and equivalents thereof.

### BRIEF DESCRIPTION OF THE DRAWINGS

FIG. 1 is a cartoon diagram of the purification process of erythrocyte membranes using a combination of biotin and streptavidin and elution with guanidine.

FIG. 2 is a figure demonstrating that the methods of purifying erythrocyte membranes are appropriate and will result in the proper identification of proteins previously demonstrated to be associated with the infected erythrocyte membrane.

FIG. 3 is a figure demonstrating the specificity of the antisera raised against the PfSA1 and PfSA2 peptides.

FIG. 4 is a figure of immunolocalization of PfSA1 and PfSA2 to the surface of P. falciparum-infected erythrocytes by confocal microscopy in two of six strains of P. falciparum tested.

FIG. 5 is a figure of immunolocalization of PfSA1 and PfSA2 to the surface of P. falciparum-infected erythrocytes with P. falciparum Malayan Camp tested where the erythrocytes had been previously treated with trypsin and chymotrypsin and in another case where the erythrocytes has been treated with trypsin and chymotrypsin in the presence of an inhibitor of trypsin and chymotrypsin

FIG. 6 is a sequence comparison of the protein sequence of PfSA1 from P. falciparum clone 3D7 against the PfSA1 sequences from three additional P. falciparum isolates (MC, R033 and 7G8).

FIG. 7 is a sequence comparison of the protein sequence of PfSA2 from P. falciparum clone 3D7 against the PfSA2 sequences from three additional P. falciparum isolates (MC, R033 and 7G8).

### DETAILED DESCRIPTION OF THE PREFERRED EMBODIMENTS

In accordance with the present invention, there is generally provided, two novel Plasmodium falciparum proteins that are expressed in association with infected human erythrocytes and these proteins are present in numerous additional strains of P. falciparum throughout the world. We have used the application of Multidimensional Protein Identification Technology (MudPIT) (Washburn et al) to analyze a mixture comprised of the P. falciparum parasitized red blood cell (PRBC) surface membrane proteins, and the identification and characterization of two novel conserved surface antigens, PfSA1 (SEQ ID NO:1) and PfSA2 (SEQ ID NO:2). In these experiments we first isolated and identified P. falciparum proteins from infected erythrocyte cultures, then raise antisera against peptide sequences from the resulting identified proteins, then confirmed the localization of the proteins near the infected erythrocyte surface, then demonstrated the protein localization on the surface of the infected erythrocytes and then determined the presence of these proteins and their variants in other P. falciparum isolates.

In a first embodiment, the invention is directed to the production of a vaccine which contains the nucleic acid sequences (SEQ ID NO:3 and SEQ ID NO: 4) or amino acid sequences (SEQ ID NO:1 and SEQ ID NO:2) of either PfSA1 or PfSA2 or both.

In a second, third and fourth embodiment this vaccine could be a recombinant protein, peptide vaccine, recombinant viral based vaccine or other vaccine delivery mechanism which when delivered by needle, needleless or ballistic injection into the body with or without adjuvants, excipients, carriers via intramuscular, intradermal, subcutaneous, intranasal, oral or other methods is designed to elicit a humoral immune response, cellular immune response or both in the human or animal in which the vaccine was administered.

In a fifth embodiment of this invention, the vaccine could be a combination of two or more of the above vaccine delivery systems, for example the delivery of three doses of a PfSA1 DNA vaccine followed by a dose of a recombinant adenovirus expressing PfSA1. The immune response against these proteins delivery by any of the means listed above, would result in a decrease in the number of Plasmodium parasites in the body, the viability of Plasmodium parasites in the body and/or the clinical manifestations of Plasmodium parasite infection. The examples of vaccines listed here are illustrative and are not meant to be exclusive.

In yet a sixth embodiment of this invention is the development of assays to detect Plasmodium parasites within the body. Antibodies are generated which react specifically with the PfSA1 or PfSA2 proteins and which would allow the development of an immunological detection assay. One example of how this would be accomplished would be to use these antibodies, alone or in combination, on biological samples taken from individuals who are suspected of being infected with Plasmodium parasites. These antibodies, for example, could be used in an Enzyme-Linked Immunosorbant Assay (ELISA) to detect the presence of PfSA1 or PfSA2 proteins in sera from patients, or in microscopic examination of blood films to detect parasites using a fluorescence-based readout. These examples are not meant to be comprehensive but only to illustrate potential uses of antibodies against PfSA1 and/or PfSA2.

A seventh embodiment of this invention is directed to the development of assays to detect Plasmodium parasites within the body based on detection of nucleic acid sequences of PfSA1 and/or PfSA2. An example of this embodiment is the use of oligonucleotide primer sequences selected from the PfSA1 and/or PfSA2 gene sequence that if used in a polymerase chain reaction assay will amplify PfSA1 and/or PfSA2 DNA or cDNA and enable the detection of the parasites by the presence of this specific nucleic acid product by gel electrophoresis, hybridization methods, or other methods known to those of skill in the art.

An eighth embodiment of this invention is directed to the identification of drugs or small molecules that can be used-as antimalarial compounds. An example of this would be the identification of a small molecule that is predicted to associate with the portion of either the PfSA1 or PfSA2 protein at the erythrocyte surface and interrupt the function of that protein with the result of causing a disruption in the Plasmodium parasite function.

The following examples are illustrative of preferred embodiments of the invention and are not to be construed as limiting the invention thereto.

### EXAMPLE 1

### Isolation of Proteins From P. falciparum Parasitized Erythrocytes

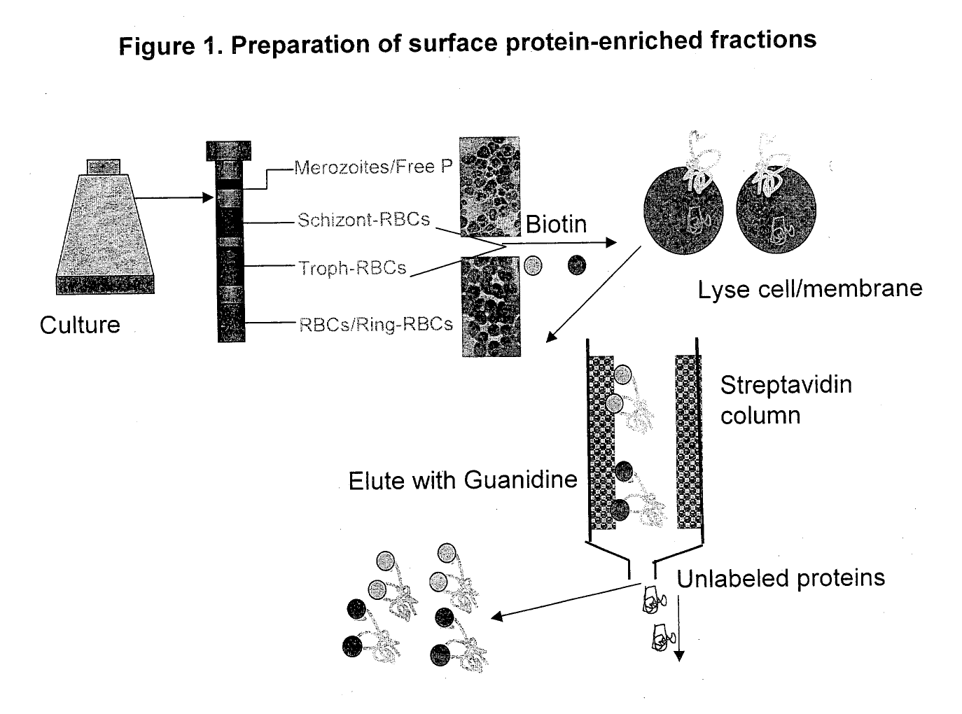

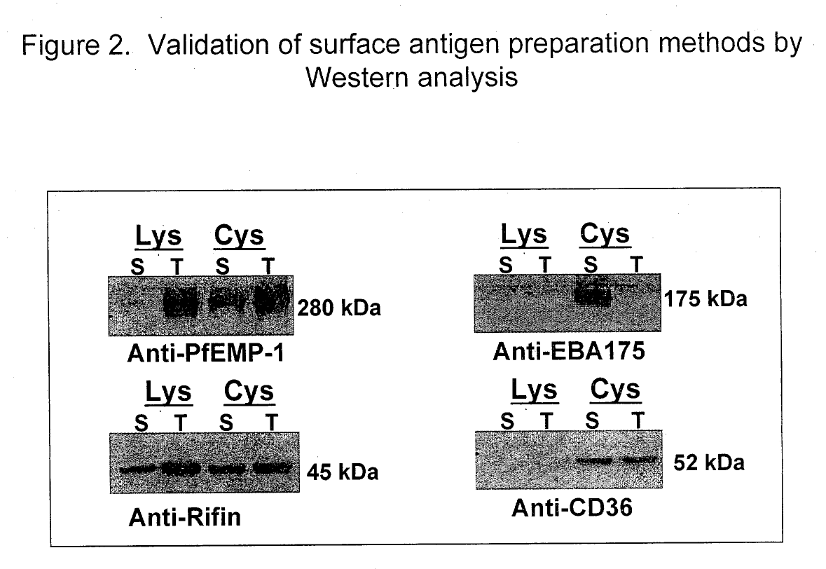

In order to obtain ALL proteins on the surface of parasitized red blood cells (PRBCs), we developed a method to label the intact PRBCs with two non-permeable biotins, Sulfo-NHS-LC-Biotin and PEO maleimide activated Biotin, with binding specificity to lysine and cystine, respectively (FIG. 1). We chose the late trophozoite-early/schizonte stage (30-36 hours post invasion, named late trophozoite stage thereafter) for the labeling because 1) an extensive surface modification was observed at this developmental stage, 2) the PRBC membrane becomes more permeable at the later developmental stage (36-48 hours post invasion, named schizont stage thereafter), which would complicate data interpretation, and 3) though not accurately quantitative, our preliminary data indicated that the cells may shed surface proteins expressed earlier (FIG. 2). After extensive washes to remove the unbound biotin protein, cells were lysed and cell debris was washed again to remove soluble proteins. Subsequently, the cell membrane was dissolved and the dissolved proteins mixture was loaded onto a streptavidin column which retains labeled proteins via biotin. Hence, the mixture eluted from the streptavidin column was enriched with surface proteins and the complexity of the sample subject to MudPIT analysis was greatly reduced. Western blotting analysis using antibodies against known surface antigens was performed to verify the extraction method (FIG. 2). Recognition of PfEMP-1, Rifin, and CD36 by specific antibodies indicates that the method effectively extracted proteins on the surface of the PRBC. The use of late trophozoite for the MudPIT analysis was supported by the observations that 1) more protein is present in the preparation from late trophozoites (30-36h post invasion) than that from the schizonts; and 2) EBA-175, a component of microneme in merozoites expressed in mature schizonts/segments, was detected in schizont stage, indicating the alteration of the membrane permeability in schizont-infected erythrocytes. In addition, CD36 was only labeled by PEO-maleimide activated biotin, suggested the necessity in using two biotins with different specificities.

### EXAMPLE 2

### Identification of P. falciparum Proteins From the Purified Parasitized Red Cell Preparation

The biotin-labeled fraction was digested with trypsin and endopeptidase C, and loaded onto biphasic microcapillary columns installed such as to spray directly into a ThermoFinnigan LCQ-Deca ion trap mass spectrometer equipped with a nano LC electrospray ionization source. Fully automated 12□step chromatography runs were carried out. SEQUEST was used to match MS/MS spectra to peptides in a sequence database combining Plasmodium falciparum and mammalian protein sequences (to account for contaminating host proteins). The validity of peptide/spectrum matches was assessed using the SEQUEST□defined parameters cross-correlation score (XCorr), Delta Cn valuer Sp rank and relative ion proportion. DTASelect (Eng, McCormack, et al 1994) was used to select and sort peptide/spectrum matches passing a conservative set of those parameters. Peptide hits from multiple runs were compared using CONTRAST (Eng, McCormack, et al 1994).

Four surface protein samples, 2 labeled with lysine-specific Sulfo-NHS-biotin and 2 with cystine-specific PEO maleimide-activated biotin, were analyzed by MudPIT. Compiling peptide hits from those 4 independent samples, 623 unique proteins were confidently identified. Among those proteins, 371 were also found in the proteomic study of whole cell lysates from P. falciparum trophozoites-schizonts (Florens, Washburn, et al 2002). Differential analysis of the sequence coverage observed for those common proteins (i.e. number of peptides leading to protein identification) allowed us to distinguish between contaminating abundant trophozoite-schizont proteins and proteins specifically enriched in the biotin-labeled fractions.

The proteins were selected for further characterization by the following criteria: 1) the presence of the signal peptide as predicted by SignalP; 2) the presence of transmembrane domain(s) as predicted by TAMP; 3) novel proteins whose function had never been characterized before; and 4) sequence conservation within multiple P. falciparum strains or/and cross Plasmodium ssp. More than 30 hypothetical proteins satisfied these criteria. Two proteins, denoted PfSA1 and PfSA2, from the 30 identified were selected for further characterization.

### EXAMPLE 3

### Bioinformatic Characterization of PfSA1 and PfSA2

The informatics package contained within a suite of informatics computer programs on the website www.plasmodb.org were used to characterize the selected proteins. Gene model prediction used GlimmerM (Salzberg, Pertea et al. 1999). PfSA1 is a hypothetical acidic protein of 1297 amino acids with theoretical molecular weight (MW) of 154 kDa and isoelectricfocusing point (IP) of 5.14. It is encoded by a single copy gene 3885 nucleotides long, denoted PfC0435w, located on P. falciparum chromosome 3 (nucleotide positions 444174-448058) and has an orthologue in P. knowlesi.

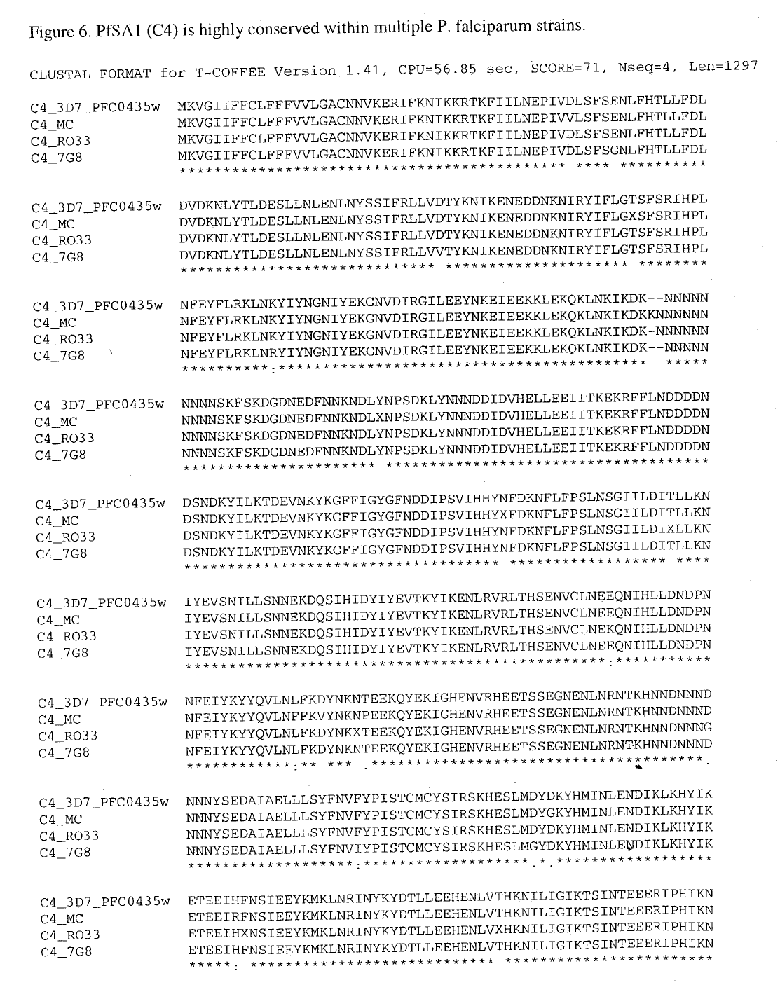

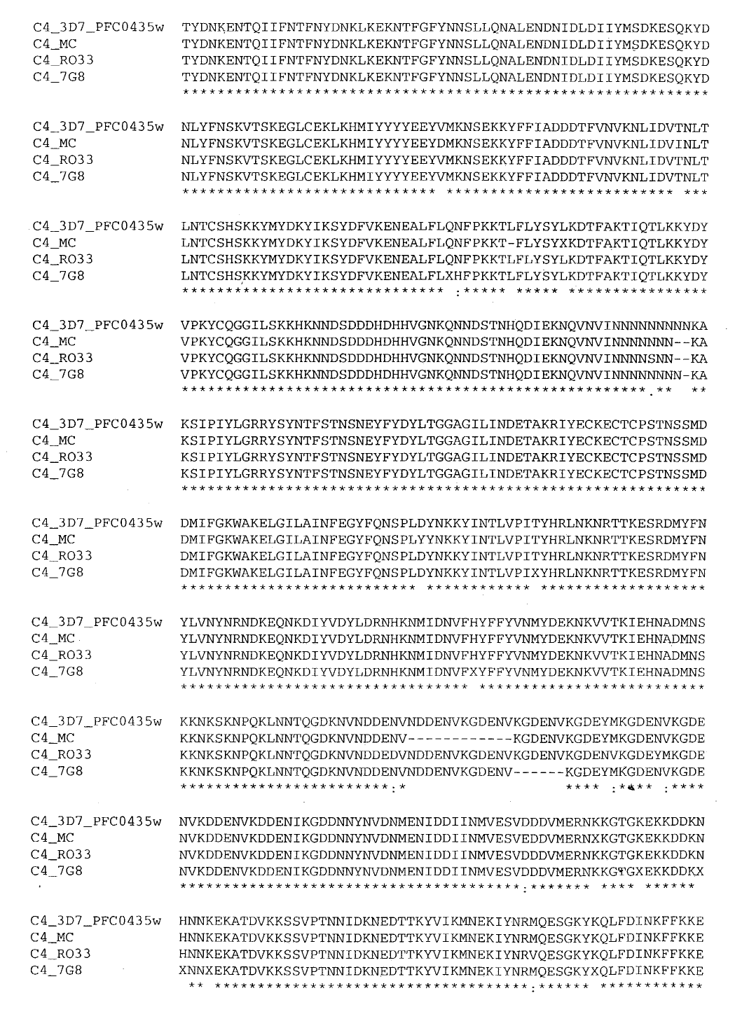

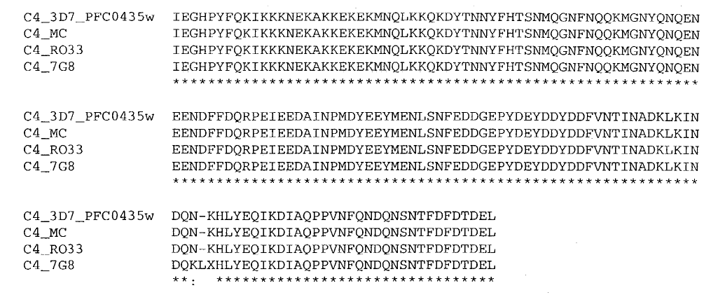

PfSA2 is a hypothetical protein of 408 amino acids with theoretical MW of 49 kDa and IP 6.67. It is encoded by a single copy two exon gene near the telomeric region of chromosome 5 (nucleotide sequences 64605-64133 and 64332-65489). It does not have discernible orthologues in other organisms (BlastP cut-off E value of 10⁻¹⁵). Both PfSA1 and PfSA2 are highly conserved in multiple strains of P. falciparum from various geographic locations (FIG. 6) suggesting their potential utility in vaccine construction.

### EXAMPLE 4

### Production of PfSA1- and PfSA2-Specific Antisera.

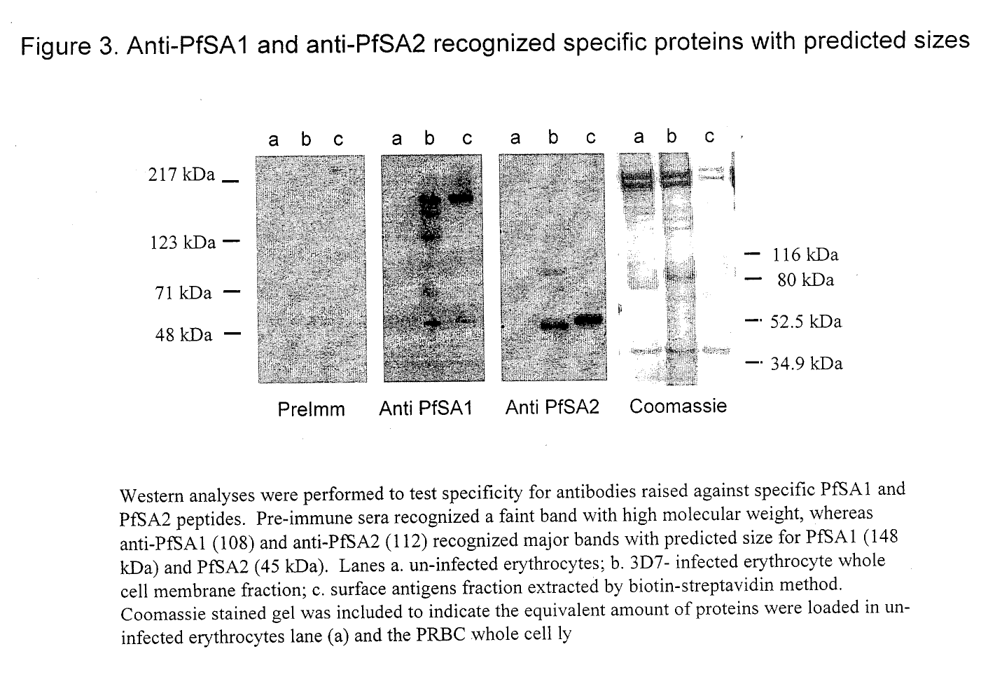

Rabbit antisera were raised against synthetic peptides designed based on PfSA1 and PfSA2. The peptide sequence used for PfSA1 is NNSKFSKDGDNEDFNNKNDLYNPSDKLYNN (SEQ ID NO:5). The peptide sequence used for PfSA2 is YEIMHKEDESKESNQHNYKEGPSYEDKKNMYKE (SEQ ID NO:6). Two specific antibodies, denoted 108 and 112, recognized proteins corresponding to the theoretical MW of PfSA1 and PfSA2, respectively, in the whole cell lysate and the biotin-labeled fraction (FIG. 3).

### EXAMPLE 5

### Localizing the Expression of PfSA1 and PfSA2 to the Erythrocyte Membrane

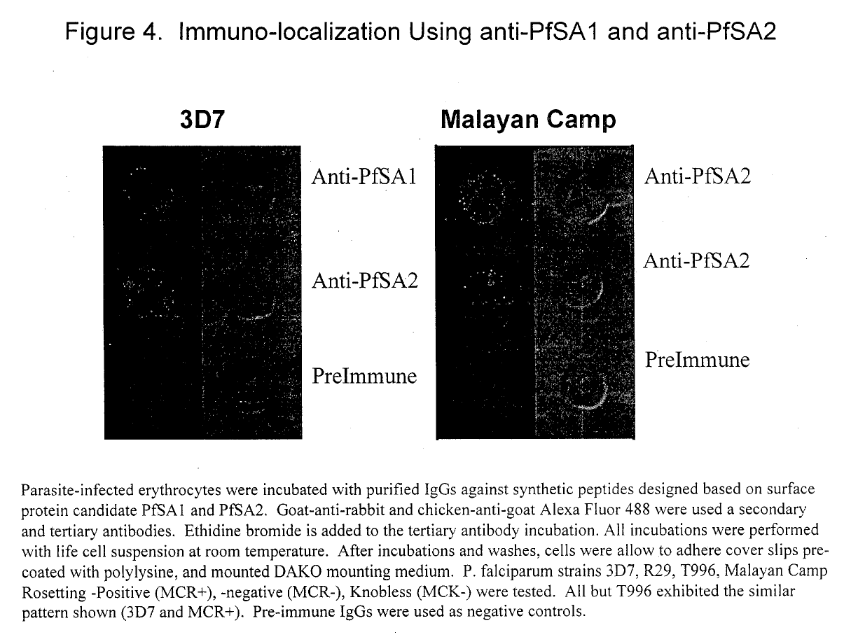

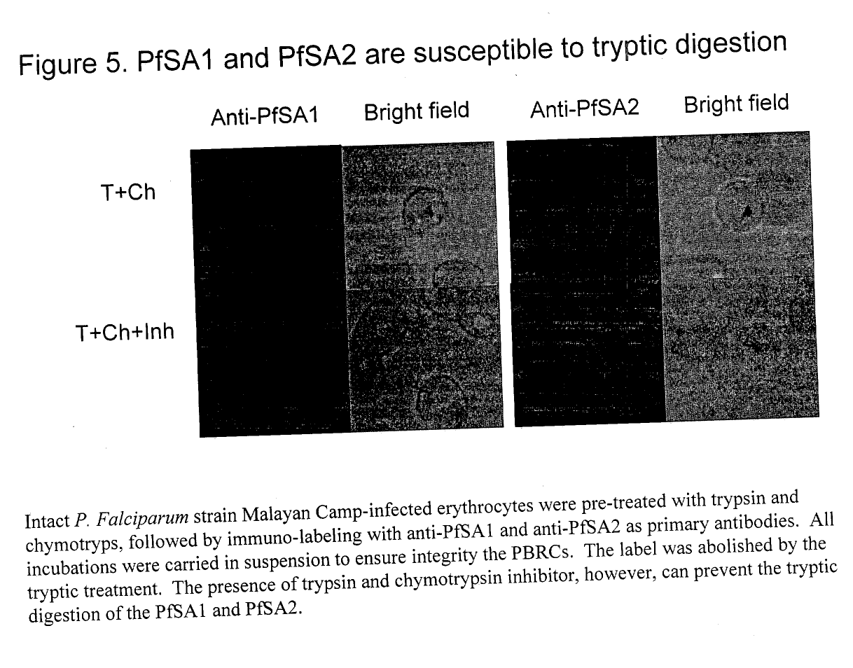

To confirm the surface location of the PfSA1 and PfSA2, we labeled the intact PRBC in suspension with purified IgG from antisera 108 and 112, followed by incubation with goat-anti-rabbit and chicken-anti-goat Alexa Fluor 488 as secondary and tertiary antibodies. Ethidium bromide was added to the incubation to stain the nuclei. The cells were allowed to adhere to cover slips pre-coated with polylysine, and examined by confocal microscopy. FIG. 4 demonstrates the localization of both antigens on the surface of PRBC. The antibody labels were abolished by pre-treating PRBCs with trypsin and chymotrypsin, confirming the surface location of the PfSA1 and PfSA2 (FIG. 5).

### EXAMPLE 6

### Further Characterizaiton of the Localization of PfSA1 and PfSA2 to Substructures on the Surface of PRBC.

The pattern of the fluorescent label with both anti-PfSA1 and anti-PfSA2 prompted us to investigate whether the antigens were part of the knobs, a protruding structure on the PRBC surface. A knobless P. falciparum strain Malayan Camp was selected for the study. Whereas the strain was verified as knobless by using an anti-KaHRP, a marker for knob structure, both anti-PfSA1 and PfSA2 were localized on the surface of the parasite, indicating the antigens were not associated with the knobs (data not shown). P. falciparum strains Malayan Camp selected for resetting positive (MCR+), and rosetting-negative (MCR−) were also tested for reactivity with anti-PfSA1 and anti-PfSA2. The antigens were present on the surface of both strains, indicating the antigens are unlikely involved in the resetting process. Of all P. falciparum strains (3D7, R29, MCR+, MCR−, MCK−, T996) test for reactivity against anti-PfSA1 and anti-PfSA2, T996 was the only one shown negative toward both antibodies (data not shown). Since PCR with primers used for sequencing PfSA1 and PfSA2 in other P. falciparum strains (see below and FIG. 6) failed to amplify any sequences from the strain T996, it is likely that the genes were deleted form the strain, or it has diverged beyond recognition. This echoes the findings that a segment of chromosome 9 was also deleted from the strain T996 (Wu, unpublished data).

### EXAMPLE 7

### Characterization of PfSA1 and PfSA2 From Other Strains of P. falciparum Parasites With Diverse World Origins.

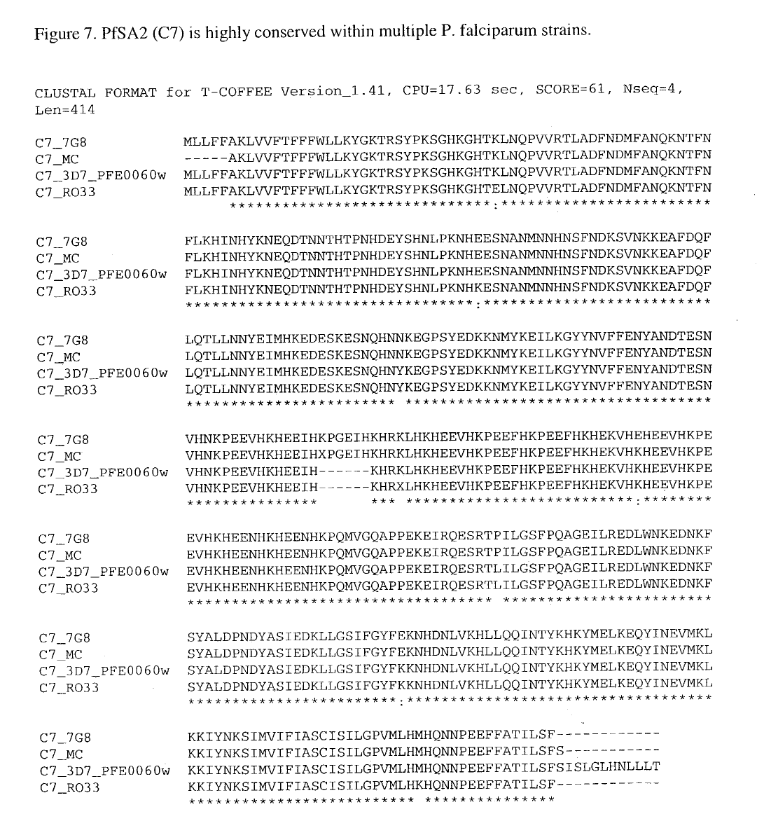

To investigate the sequence conservation of PfSA1 and PfSA2, specific primers were designed to amplify and sequence the antigens from the selected P. falciparum isolates from various geographic location, 7G8 (South America), Malayan Camp (MC) (Southeast Asia), and R033 (Africa). As shown in FIGS. 6 and 7, both proteins are remarkably conserved with other P. falciparum strains, indicating both could be good vaccine candidates with broad specificity.

This is the first study applying high throughput proteomics approach toward the identification of proteins on the surface of PRBCs. The method is highly efficient because, of two antigens selected for detailed characterization, both were confirmed to be on the surface of PRBCs. Further evaluation on immunogenicity of PfSAl and PfSA2 and efficacy of anti-PfSA1 and anti-PfSA2 will provide insight whether the antigens can be targets for antimalarial vaccines. Our findings also indicate that the surface composition of PRBC is more complex than we thought, as more candidates as result of our in silico analysis awaits to be analyzed and are also likely to be surface proteins. Some of these proteins might be account for the protective immunity, some might mediate cytoadherence, yet some might be channels responsible nutrient uptake.

### PROPHETIC EXAMPLE 8

### Development of a PfSA1 Malaria Vaccine

In this example, a DNA vaccine encoding the full length of PfSA1 or PfSA2 is produced under GMP and is delivered in three doses intramuscularly at 5 milligrams per dose at monthly intervals, to be followed by a recombinant adenovirus vaccine which is designed to express PfSA1 or PfSA2 and which is delivered at dose of 10exp11 viral particles intramuscularly one month after the last dose of DNA vaccine. In another example, a recombinant adenovirus vaccine which is designed to express PfSA1 is delivered in two or three doses at one month intervals at a dose of l0expli viral particles per dose intramuscularly. In these examples, these vaccines could be used alone in a population of children living in SubSaharan Africa to reduce the number of circulating Plasmodium infected erythrocytes and would result in a decrease in morbidity and mortality associated with malaria. These vaccines could also be used in combination with other vaccines which are directed against the liver stages of the parasite to limit the risk of developing severe malaria in those individuals where the liver stage vaccines are less than 100% effective.

### PROPHETIC EXAMPLE 9

### Development of a Rapid Assay to Detect Plasmodium Infection in Humans

In this example, polyclonal or monoclonal antibodies raised against polypeptide sequences from PfSA1 or PfSA2 can be used in an immunologic based assay to detect circulating PfSA1 and/or PfSA2 in serum, or to assist in the identification of parasite-infected erythrocytes in blood smears from patients suspected of being infected with Plasmodium. In these examples, the readout could be an enzyme linked immunosorbant assay, a fluorescence-based assay or a calorimetric based assay, though other means of assessing the detection of parasites using these antibodies may also be employed.

### PROPHETIC EXAMPLE 10

### Method for the Detection of Additional Plasmodium Proteins From the Surface of Plasmodium-Infected Erythrocytes

In this example, additional Plasmodium proteins that are located on the surface of infected erythrocytes are detected by a similar means as described above. These proteins would represent novel proteins for vaccine development as their location on the surface of infected-erythrocytes predicts that they will encounter cells of the immune system which will respond with the production of a humoral and/or cellular immune response against erythrocyte infected with Plasmodium. These additional proteins and the gene sequences encoding for these proteins can be used as vaccines delivered by DNA vaccine, recombinant protein, recombinant viral vaccine or other vaccine delivery systems.

### PROPHETIC EXAMPLE 11

### Development of a PfSA1 or PfSA2 recombinant protein malaria vaccine In this example, the DNA sequence of PfSA1 or PfSA2 is cloned into a bacterial expression system and a purified recombinant PfSA1 or PfSA2 protein is purified under cGMP and delivered at a dose of 50 micrograms intramuscularly at one month intervals for three months. In this example, antibodies against the PfSA1 or PfSA2 proteins will be produced will react with these proteins on the surface of the infected erythrocyte and result in the elimination of the infected erythrocyte from the circulation.

### References

Altschul S F, Gish W, Miller W, Myers E W, Lipman D J. Basic local alignment search tool. J Mol Biol Oct. 5, 1990;215(3):403-10

Chen, Q., V. Fernandez, et al. (1998). “Developmental selection of var gene expression in Plasmoodium falciparum.” Nature 394(6691): 392-5.

Eng, J. K., A. L. McCormack, et al. (1994). “An approach to correlate tandem mass spectral data of peptides with amino acid sequences in a protein database.” Journal of the American Society for Mass Spectrometry 5: 976-989.

Fernaders (1998). J Exp Med 190: 1393-1404.

Florens, L., M. P. Washburn, et al. (2002). “A proteomic view of the Plasmodium falciparum life cycle.” Nature 419(6906): 520-6.

Hollingdale, M. R., C. J. McCormick, et al. (1998). “Biology of malarial liver stages: implications for vaccine design.” Ann. Trop. Med. Parasitol. 92: 411-417.

Howard (1988). Prog. Allergy 41: 98-147.

Kyes (1999). Proc natl Acad Sci USA 96: 9333-9338.

McGregor (1963). Trans. R. Soc. Tropo. Med. Hyg 57: 170-175.

McGregor and Wilson (1988). Principles and Practices of Malaria. Malaria. Wensdorfer.

Mohan, B. N. and Stevenson (1998). Pathogenesis and Protection. Malaria Parasite Biology. Sherman.

Preiser, P., M. Kaviratne, et al. (2000). “The apical organelles of malaria merozoites: host cell selection, invasion, host immunity and immune evasion.” Microbes Infect 2(12): 1461-77.

Richie, T. L. and A. Saul (2002). “Progress and challenges for malaria vaccines.” Nature 415(6872): 694-701.

Salzberg, S. L., M. Pertea, et al. (1999). “Interpolated Markov models for eukaryotic gene finding.” Genomics 59(1): 24-31.

Washburn, M. P., D. Wolters, et al. (2001). “Large-Scale Analysis of the Yeast Proteome via Multidimensional Protein Identification Technology.” Nat Biotechnol.

The inventive subject matter being thus described, it will be obvious that the same may be varied in many ways. Such variations are not to be regarded as a departure from the spirit and scope of the inventive subject matter, and all such modifications are intended to be within the scope of the following claims.

What is claimed is:

1. An immunogenic composition of Plasmodium proteins comprising:
an immunogenic protein or polypeptide selected from the group comprising SEQ ID NO:l, SEQ ID NO:2, sequences homologous to SEQ ID NO: 1 or SEQ ID NO: 2 as defined as having greater than 80% sequence identity, and
combinations thereof, wherein administration of said composition elicits an immune response to the whole or part of the said proteins or polypeptides.

2. The immunogenic composition of claim 1, wherein polypeptide fragments containing B-cell epitopes induce antibodies which react with SEQ ID NQ:1 or SEQ ID NO:2.

3. The immunogenic composition of claim 1, wherein polypeptide fragments containing T-cell epitopes induce cellular immune responses against SEQ ID NO:1 or SEQ ID No:2:

4. The polypeptide of claim 1, wherein said polypeptide is SEQ ID NO:5.

5. The polypeptide of claim 1, wherein said polypeptide is SEQ ID NO:6.

6. The immunogenic composition of claim 1, wherein said Plasmnodium proteins are native or recombinantly expressed.

7. The immunogenic composition of claim 6, further comprising a recombinant viral vaccine, wherein said immunogenic proteins or polypeptides are expressed.

8. The immunogenic composition of claim 7, wherein said recombinant virus is an adenovirus.

9. The immunogenic composition of claim 7, wherein said recombinant virus is a vaccinia virus.

10. The immunogenic composition of claims 1, wherein said Plasmodium protein or polypeptide is derived from the species consisting of P. vivax, P. ovale and P. malariae.

11. An immunogenic composition comprising:
a nucleic acid sequence encoding an immunogenic protein or polypeptide selected from the group comprising SEQ ID NO:3, SEQ ID NO:4, sequences with 80% sequence identity to SEQ ID NO: 3 or SEQ ID NO: 4, and combinations thereof, wherein administration of said composition elicits an immune response.

12. The immunogenic composition of claim 11, wherein said nucleic acid sequence is inserted into a DNA vaccine plasmid and wherein polypeptide fragments are expressed containing B-cell epitopes that induce antibodies which react with SEQ ID NO:1 or SEQ ID NO:2.

13. The immunogenic composition of claim 11, wherein said nucleic acid sequence is inserted into a recombinant virus and wherein polypeptide fragments are expressed containing B-cell epitopes that induce antibodies which react with SEQ ID NO:1 or SEQ ID NO:2.

14. The immunogenic composition of claim 11, wherein said composition is administered in addition to a recombinant virus expressing the amino acid sequence of SEQ ID NO:1 or SEQ ID NO:2.

15. A method to detect the presence of Plasmodium parasite through the identification of proteins PfSA1 or PfSA2, the method comprising:
a. obtaining a sample from a patient suspected of infection;
b. contacting said sample with polyclonal or monoclonal antibodies to all or portions of SEQ ID NO:1 or SEQ ID NO:2 or a combination thereof in an Enzyme-Linked Immunosorbent Assay or other immunological based method;
c. assessing the reactivity of the applied antibodies by visualization; and
d. assessing the presence of parasites by enhanced visualization under microscopy.

16. A method to detect the presence of Plasmodium parasite through the identification of proteins PfSA1 or PfSA2, the method comprising:
a. obtaining a sample from a patient suspected of infection
b. preparing genomic DNA or cDNA from the patient sample.
c. subjecting the genomic DNA or cDNA to polymerase chain reaction involving oligonucleotide primers from either the nucleotide sequences of SEQ ID NO:3 or SEQ ID NO:4.
d. visualizing the presence of specific amplified product by assay methods.

17. The method of claim 16, wherein said sample is obtained from blood or tissues.

18. An isolated antibody or portion thereof that specifically binds to a protein consisting of an amino sequence of SEQ ID NO:1 or SEQ ID NO:2.
